(19) United States
(12) Patent Application Publication
(10) Pub. No.: US 2010/0316730 A1
(43) Pub. Date: Dec. 16, 2010

(54) TREATMENT OF CARDIOVASCULAR DISEASES WITH OZONE

(76) Inventors: Joseph S. Latino, Brooklyn, NY (US); Steven A. Keyser, Salt Lake City, UT (US)

Correspondence Address: MORRISS OBRYANT COMPAGNI, P.C., 734 EAST 200 SOUTH, SALT LAKE CITY, UT, 84102, US

(21) Appl. No.: 12/819,875

(22) Filed: Jun. 21, 2010

Related U.S. Application Data
(63) Continuation-in-part of application No. 12/813,371, filed on Jun. 10, 2010.
Application No. 12/813,371 is a division of application No. 10/963,477, filed on Oct. 11, 2004, now Pat. No. 7,736,494.
Application No. 10/963,477 is a continuation-in-part of application No. 10/910,485, filed on Aug. 2, 2004, now abandoned.
(60) Provisional application No. 61/269,091, filed on Jun. 19, 2009.
(60) Provisional application No. 60/553,774, filed on Mar. 17, 2004.
(60) Provisional application No. 60/491,997, filed on Jul. 31, 2003.

(51) Int. Cl. A61K 33/00; A61P 9/10; A61P 9/12; A61P 9/04; A61P 9/00

(52) U.S. Cl. 424/613

(57) ABSTRACT

Methods of treating cardiovascular diseases, including atherosclerosis, peripheral arterial occlusive disease, congestive heart failure, hypertension, cerebrovascular disease, dyslipidemia and vasospastic disorders such as Raynaud's disease, in a mammalian patient involve extracorporeally subjecting an amount of blood, blood fractionate or other biological fluid from a patient to a measured amount of ozone delivered by an ozone delivery system, resulting in the absorption of a quantifiable absorbed-dose of ozone and reinfusing the treated fluid into the patient to therapeutically treat cardiovascular disease and related conditions.

### CROSS REFERENCE TO RELATED APPLICATIONS

This is a non-provisional application claiming priority to provisional Ser. No. 61/269,091, filed Jun. 19, 2009, and this non-provisional application also claims priority to co-pending Ser. No. 12/813,371, filed Jun. 10, 2010, which is a divisional application of Ser. No. 10/963,477, filed Oct. 11, 2004, which is a continuation-in-part of Ser. No. 10/910,485, filed Aug. 2, 2004, which claims priority to both provisional application Ser. No. 60/553,774, filed Mar. 17, 2004, and provisional application Ser. No. 60/491,997, filed Jul. 31, 2003. The contents of all foregoing applications are incorporated herein, in their entirety, by reference.

### BACKGROUND OF THE INVENTION

1. Field of the Invention

This invention relates to therapeutic treatments for cardiovascular diseases including atherosclerosis, peripheral arterial occlusive disease, congestive heart failure, hypertension, cerebrovascular disease, dyslipidemia, and vasospastic disorders, including Raynaud's disease, in a mammalian patient, and specifically relates to therapeutic treatment of cardiovascular disorders or conditions using quantifiable absorbed doses of ozone delivered to a biological fluid by an ozone delivery system.

2. Statement of the Relevant Art

The references discussed herein are provided solely for the purpose of describing the field relating to the invention. Nothing herein is to be construed as an admission that the inventors are not entitled to antedate a disclosure by virtue of prior invention. Furthermore, citation of any document herein is not an admission that the document is prior art, or considered material to patentability of any claim herein, and any statement regarding the content or date of any document is based on the information available to the applicant at the time of filing and does not constitute an affirmation or admission that the statement is correct.

Cardiovascular diseases are responsible for a significant number of deaths in most industrialized countries. One such disease is atherosclerosis, a disease of large and medium-sized muscular arteries and is characterized by endothelial dysfunction, vascular inflammation, and the buildup of lipids, cholesterol, calcium, and cellular debris within the intima of the vessel wall. This buildup results in plaque formation, vascular remodeling, acute and chronic luminal obstruction and abnormalities of blood flow, and also results in ischemia (diminished oxygen supply to organs and tissues) of target organs such as the heart, brain and other vital organs. Prolonged or sudden ischemia may result in a clinical heart attack or stroke from which the patient may or may not recover.

The true frequency of atherosclerosis is difficult, if not impossible, to accurately determine because it is predominantly an asymptomatic condition. The process of atherosclerosis begins in childhood with the development of fatty streaks and advances with increasingly more complicated lesion formation throughout adult life.

In the United States, approximately 7.8 million myocardial infarctions occur annually, and more than 13.2 million Americans have chronic coronary artery disease. Of persons older than 50 years, 30% have some evidence of carotid artery disease, and cerebrovascular disease is responsible for over 160,000 deaths per year in the United States. More than 50 million people in the United States are candidates for some form of dietary and/or drug treatment to modify their lipid profile.

Pathophysiology: A complex and incompletely understood interaction exists between the critical cellular elements of the atherosclerotic lesion. These cellular elements include endothelial cells, smooth muscle cells, platelets, and leucocytes. Vasomotor function, the thrombogenicity of the blood vessel wall, the state of activation of the coagulation cascade, the fibrinolytic system, smooth muscle cell migration and proliferation, and cellular inflammation are complex and interrelated biological processes that contribute to atherogenesis and the clinical manifestations of atherosclerosis.

The mechanisms of atherogenesis remain uncertain. It is presently believed that early events include endothelial injury, which cause vascular inflammation and a fibroproliferative response ensues.

The earliest pathologic lesion of atherosclerosis is the fatty streak and is observed in the aorta and coronary arteries of most individuals by age 20 years. The fatty streak is the result of localized accumulation of serum lipoproteins within the intima of the vessel wall. The fatty streak may progress to form a fibrous plaque, and is the result of progressive lipid accumulation and the migration and proliferation of smooth muscle cells. Activators of cell-division are produced by activated platelets, macrophages and dysfunctional endothelial cells that characterize early atherogenesis, vascular inflammation, and platelet-rich thrombosis at sites of endothelial disruption.

Vascular inflammation, believed to be a significant component in the etiology of atherosclerosis, may be due to an imbalance between pro-inflammatory (e.g. interferon-gamma, TNF-gamma, IL-6, IL-8 and IL-12) and anti-inflammatory cytokine (e.g. interleukin-4 and IL-10) release by immunomodulatory T cells associated with an atherosclerotic lesion. Similar imbalances have been implicated in other autoimmune diseases such as psoriasis, rheumatoid arthritis, scleroderma, lupus, diabetes mellitus, organ rejection, miscarriage, multiple sclerosis, inflammatory bowel disease as well as graft versus host disease.

There is also an emerging body of literature which indicates that the vascular endothelium plays a major role in the regulation of blood flow through the cardiovascular system and is of importance in connection with cardiovascular disorders. A dysfunctional endothelium has been suggested as a contributory factor in many cardiovascular diseases such as atherosclerosis, peripheral arterial occlusive disease and many other circulatory disorders observed in mammalian patients. Recent evidence indicates that a relative deficiency in endothelium-derived nitric oxide, a vasodilator, further potentiates the proliferative stage of plaque maturation.

Growth of the fibrous plaque results in vascular remodeling, progressive luminal narrowing, blood-flow abnormalities, and compromised oxygen supply to target organs. Human coronary arteries enlarge in response to plaque formation, and luminal stenosis may only occur once the plaque occupies greater than 40% of the area bounded by the internal elastic lamina.

The stripping or removal (i.e. denudation) of the overlying endothelium or rupture of the protective fibrous cap may result in exposure of the thrombogenic contents of the core of the plaque to the circulating blood. A plaque rupture may result in thrombus formation, partial or complete occlusion of the blood vessel, and progression of the atherosclerotic lesion due to organization of the thrombus and incorporation within the plaque.

Physical Symptoms and Clinical Events of Atherosclerosis: The physical symptoms of atherosclerosis provide objective evidence of extracellular lipid deposition, stenosis or dilatation of large muscular arteries, or target organ ischemia or infarction, and include the physical symptoms discussed hereinafter.

Intermittent claudication: Claudication, which is defined as reproducible ischemic muscle pain, is one of the most common manifestations of peripheral arterial occlusive disease caused by atherosclerosis. Claudication occurs during physical activity and is relieved after a short rest. Calf, thigh or buttock pain develops because of inadequate blood flow. The most feared consequence of claudication is severe limb-threatening ischemia leading to amputation. However, studies of large patient groups with claudication reveal that amputation is uncommon. Intermittent claudication may be accompanied by pallor of the extremity and paresthesias (abnormal sensation, such as tingling or burning of touch without stimulus).

Intermittent claudication typically causes pain that occurs with physical activity. Determining how much physical activity is needed before the onset of pain is crucial. Typically, vascular surgeons relate the onset of pain to a particular walking distance in terms of street blocks (e.g. two-block claudication). This helps to quantify patients with some standard measure of walking distance before and after therapy. Other important aspects of claudication pain are that the pain is reproducible within the same muscle groups and that it ceases with a resting period of 2-5 minutes. Location of the pain is determined by the anatomical location of the arterial lesions.

Additional muscular symptomology; Reduced blood flow that may be caused by either cholesterol embolism or arterial stenosis is frequently associated with muscular symptomology in an extremity or muscle group distal to the embolism or vascular constriction. Numbness and tingling, muscular spasm, weakness and loss of movement are common reportable events.

Extremity temperature: Reduced flow of blood resulting in oxygen deprivation to an organ or tissue (ischemia) is commonly associated with both atheroembolism (cholesterol embolism) and arterial stenosis. This is frequently associated with a measurable decrease the temperature of an extremity distal to the site of the embolism or vascular narrowing.

Decreased pulse: A decrease or loss of pulse due to reduced blood flow in instances of atheroembolism and arterial stenosis is a quantifiable parameter and frequently associated with loss of pallor in an extremity.

Hypertension secondary to arterial stenosis: The primary factor in hypertension is an increase in peripheral resistance resulting from vasoconstriction of peripheral blood vessels secondary to arterial stenosis.

Weight gain: A major factor underlying weight gain is lipid deposition secondary to the accumulation of excessive triglycerides or the inhibition in the clearance of triglycerides.

Clinical events relating to cardiovascular disease include progressive luminal narrowing of an artery due to expansion of a fibrous plaque, which results in impairment of flow when more than 50-70% of the lumen diameter is obstructed. This impairment in flow results in symptoms of inadequate blood supply to a target organ in the event there is an increase in metabolic activity and therefore oxygen demand. Stable angina pectoris, intermittent claudication, and mesenteric angina are examples of the clinical consequences of this condition.

Rupture of a plaque or denudation of the endothelium overlying a fibrous plaque may result in exposure of the highly thrombogenic subendothelium and lipid core. This exposure may result in thrombus formation, which may partially or completely occlude flow in the involved artery. Unstable angina pectoris, myocardial infarction, transient ischemic attack, and stroke are examples of the clinical manifestations of partial or complete acute occlusion of an artery.

Atheroembolism, also known as cholesterol embolism, refers to the occlusion of small- and medium-caliber arteries (100-200 μm in diameter) by cholesterol crystals. It may present with symptoms of digital necrosis, hypertension, gastrointestinal bleeding, myocardial infarction, retinal ischemia, cerebral infarction, and renal failure. Physical signs include Livedo reticularis (a persistent purplish network-patterned discoloration of the skin caused by dilation of capillaries and venules due to stasis or changes in underlying blood vessels), gangrene, cyanosis, and ulceration. The presence of pedal pulses in the setting of peripheral ischemia suggests microvascular disease.

Angina pectoris is characterized by retrosternal chest discomfort that typically radiates to the left arm and may be associated with dyspnea. Angina pectoris is exacerbated by exertion and relieved by rest or nitrate therapy. Unstable angina pectoris describes a pattern of increasing frequency or intensity of episodes of angina pectoris and includes pain at rest. A prolonged episode of angina pectoris that may be associated with diaphoresis is suggestive of myocardial infarction.

Cerebrovascular disease designates any abnormality of the brain resulting from a pathologic process of the blood vessels, e.g. occlusion of the lumen by a thrombus or embolus, rupture of a vessel, any lesion or altered permeability of the vessel wall and increased viscosity or other change in quality of blood. Disorders of the cerebral circulation include any disease of the vascular system that causes ischemia or infarction of the brain or spontaneous hemorrhage into the brain or subarachnoid space.

A cerebrovascular accident (CVA) or stroke is the sudden death of brain cells due to impaired blood flow resulting in abnormal brain function. Blood flow to the brain can be disrupted by either a blockage (clogging of arteries within the brain, carotid arterial occlusion or embolism) or rupture of an artery (cerebral hemorrhage or subarachnoid hemorrhage) to the brain.

A transient ischemic attack (TIA) is a short-lived episode (less than 24 hours) of temporary impairment of the brain that is caused by a loss of blood supply. A TIA causes a loss of function in the area of the body that is controlled by the portion of the brain affected.

Causative factors involved in cerebrovascular disease includes cerebral infarction and ischemia which is caused by sudden occlusion of an artery supplying the brain, or, less often, by low flow distal to an already occluded or highly stenosed artery. Occlusion or stenosis can be the result of disease of the arterial wall or embolism from the heart. Infarction originates as a result of an impediment to normal perfusion that usually is caused by atherosclerosis and coexisting thrombosis. Atheroembolism (atheroma) occurs when a particle of a thrombus originating from a proximal source (arterial, cardiac or transcardiac) travels through the vascular system and leads to a distal occlusion.

A corollary and additional factor in cerebrovascular disease is the incidence of intracranial small vessel disease (microatheroma). The small penetrating arteries of the brain are not supported by a good collateral circulation and occlusion of one of these arteries is rather likely to cause infarction, often in a small, restricted area of the brain.

Inflammatory vascular disease of the arterial (or venous) wall may provoke enough cellular proliferation, necrosis and fibrosis to occlude the lumen, precipitate thrombosis and then embolism, or promote aneurysm formation, dissection and even rupture of the vessel. These vasculitic disorders may present with, or be complicated during their course by, ischemic stroke, intracranial hemorrhage, intracranial venous thrombosis and, most often, a generalized ischemic encephalopathy.

Physical signs of cerebrovascular disease include diminished carotid pulses, carotid artery bruits, and focal neurological deficits.

Peripheral arterial occlusive disease (PAOD) typically manifests as intermittent claudication, impotence, and non-healing ulceration and infection of the extremities. PAOD is most common with the distal superficial femoral artery (located just above the knee joint), which corresponds to claudication in the calf muscle area (the muscle group just distal to the arterial disease). When atherosclerosis is distributed throughout the aortoiliac area, thigh and buttock muscle claudication predominates.

Physical signs include decreased peripheral pulses, peripheral arterial bruits (an unexpected audible swishing sound or murmur heard over an artery or vascular channel which indicates increased turbulence often caused by a partial obstruction), pallor, peripheral cyanosis, gangrene, ulceration. Visceral ischemia may be occult or symptomatic prior to symptoms and signs of target organ failure.

Mesenteric angina is characterized by epigastric or periumbilical postprandial pain and may be associated with hematemesis, melena, diarrhea, nutritional deficiencies, and weight loss. Abdominal aortic aneurysm typically is asymptomatic prior to the dramatic and often fatal symptoms and signs of rupture, although patients may describe a pulsatile abdominal mass. Physical signs include pulsatile abdominal mass, peripheral embolism and circulatory collapse.

Dyslipidemia is a disorder of lipoprotein metabolism, including lipoprotein overproduction or deficiency. Dyslipidemias may be manifested by elevation of the total cholesterol, low-density lipoprotein (LDL) cholesterol and the triglyceride concentrations, and a decrease in the high-density lipoprotein (HDL) cholesterol concentration in the blood.

Congestive Heart Failure (CHF), most frequently resulting from coronary artery disease or hypertension, and occurs when the heart can no longer meet the metabolic demands of the body at normal physiologic venous pressures. As the demands on the heart outstrip the normal range of physiologic compensatory mechanisms, signs of CHF occur. These signs include tachycardia, venous congestion, high catecholamine levels and, ultimately, insufficient cardiac output. Chronic inflammation is recognized as an underlying pathology contributing to the development and progression of chronic heart failure.

Raynaud's disease refers to a disorder in which the fingers or toes (digits) suddenly experience decreased blood circulation. Raynaud's disease can be classified as either primary (or idiopathic) and secondary (also called Raynaud's phenomenon). Primary Raynaud's disease is milder, and causes fewer complications.

Secondary Raynaud's disease is more complicated, severe, and more likely to progress. A number of medical conditions predispose a person to secondary Raynaud's disease, including scleroderma, which is a serious disease of the connective tissue in which tissues of the skin, heart, esophagus, kidney and lung become thickened, hard and constricted. About 30% of patients who develop scleroderma will first develop Raynaud's disease. Other medical conditions predisposing a person to secondary Raynaud's disease include connective tissue diseases, such as systemic lupus erythematosus, rheumatoid arthritis, dermatomyositis and polymyositis, and diseases which result in blockages of arteries (i.e. atherosclerosis).

Both primary and secondary types of Raynaud's symptoms are believed to be due to over-reactive arterioles (small arteries). While cold normally causes the muscle which makes up the walls of arteries to contract, in Raynaud's disease the degree is extreme, and blood flow to the area is severely restricted.

The relationship between dietary lipid, serum cholesterol and atherosclerosis has long been recognized. In many epidemiological studies it has been shown that a single measurement of serum cholesterol has proved to be a significant predictor of the occurrence of coronary heart disease. Thus diet is the basic element of all therapy for hyperlipidemia (excessive amount of fat in plasma). However, the use of diet as a primary mode of therapy requires a major effort on the part of physicians, nutritionists, dietitians and other health professionals. If dietary modification is unsuccessful, drug therapy is an alternative. Several drugs, used singly or in combination, are available. However, there is no direct evidence that any cholesterol-lowering drug can be safely administered over an extended period.

A combination of both drug and diet may be required to reduce the concentration of plasma lipids. Hypolipidemic drugs are therefore used as a supplement to dietary control. Many drugs are effective in reducing blood lipids, but none work in all types of hyperlipidemia and they all have undesirable side effects. There is no conclusive evidence that hypolipidemic drugs can cause regression of atherosclerosis. Thus, despite progress in achieving the lowering of plasma cholesterol to prevent heart disease by diet, drug therapies, surgical revascularization procedures and angioplasty, atherosclerosis remains the major cause of death in Western countries.

In view of the above, new approaches are being sought to reduce the frequency of clinical sequelae secondary to the myriad of diseases and disorders broadly characterized as cardiovascular diseases.

### Apoptosis

Apoptosis specifically refers to an energy-dependent, asynchronous, genetically controlled process by which unnecessary or damaged single cells self-destruct when apoptosis genes are activated (Martin, S J 1993; Earnshaw, W C 1995). There are three distinct phases of apoptosis. Initially, the cell shrinks and detaches from neighboring cells. The nucleus is broken down with changes in DNA including strand breakage (karyorhexis) and condensation of nuclear chromatin (pyknosis). In the second phase, nuclear fragments and organelles condense and are ultimately packaged in membrane-bound vesicles, exocytosed and ingested by surrounding cells. In the final phase, membrane integrity is finally lost and permeability to dyes (i.e. trypan blue) occurs. The absence of inflammation differentiates apoptosis from necrosis when phagocytized by macrophages and epithelial cells (Kam, PCA 2000).

In contrast, necrotic cell death is a pathological process caused by overwhelming noxious stimuli (Lennon, S V 1991). Synchronously occurring in multiple cells, it is characterized by cell swelling or “oncosis,” resulting in cytoplasmic and nuclear swelling and an early loss of membrane integrity. Bleb formation (blister-like, fluid filled structures) of the plasma membrane occurs, in which ultimate rupture may occur causing an influx of neutrophils and macrophages in the surrounding tissue, and leading to generalized inflammation (Majno, G 1995).

Four main groups of stimuli for apoptosis have been reported; ionizing radiation and alkylating anticancer drugs causing DNA damage, receptor mechanism modulation (i.e. glucocorticoids, tumor necrosis factor-α, nerve growth factor or interleukin-3), enhancers of apoptotic pathways (i.e. phosphatases and kinase inhibitors), and agents that cause direct cell membrane damage and include heat, ultraviolet light and oxidizing agents (i.e. superoxide anions, hydroxyl radicals and hydrogen peroxide) (Kam, PCA 2000).

In addition to the oxidizing agents, many chemical and physical treatments capable of inducing apoptosis are also known to evoke oxidative stress (Buttke, M 1994, Chandra, J 2000). Ionizing and ultraviolet radiation both generate reactive oxygen intermediates (ROI) such as hydrogen peroxide and hydroxyl free radicals. Low doses of hydrogen peroxide (10-100 μM) induces apoptosis in a number of cell types directly establishing oxidative stress as a mediator of apoptosis. However, high doses of this oxidant induce necrosis, consistent with the concept that the severity of the insult determines the form of cell death (apoptosis vs, necrosis) that occurs. A free radical is not a prerequisite for inducing apoptosis; doxorubucin, cisplatin and ether-linked lipids are anti-neoplastics that induce apoptosis and oxidative damage.

Alternatively, oxidative stress can be induced by decreasing the ability of a cell to scavenge or quench reactive oxygen intermediates (ROI) (Buttke, M 1994). Drugs (i.e. butathionine sulfoxamine) that reduce intracellular glutathione (GSH) render cells more susceptible to oxidative stress-induced apoptosis. Cell studies report a direct relationship between extracellular catalase levels and sensitivity to hydrogen peroxide-induced apoptosis. Apoptosis induced through tumor necrosis factor-α stimulation has been demonstrated to be associated with an increase in intracellular ROI. However, this apoptosis has been inhibited by the addition of a number of antioxidants, such as thioredoxin, a free radical scavenger, and N-acetylcysteine, an antioxidant and GSH precursor.

There is growing evidence that apoptotic neutrophils have an active role to play in the regulation and resolution of inflammation following phagocytosis by macrophages and dendritic cells. A hallmark of phagocytic removal of necrotic neutrophils by macrophages is an inflammatory response including the release of proinflammatory cytokines (Vignola, A M 1998, Beutler, B 1988, Moss, S T 2000, Fadok V A, 2001). In contrast, apoptotic neutrophil clearance is not accompanied by an inflammatory response; phagocytosis of these apoptotic cells has been shown to inhibit macrophage production of pro-inflammatory cytokines (GM-CSF, IL-1β, IL-8, TNF-α, TxB2, and LTC4) with a concomitant activation of anti-inflammatory cytokine production (TGF-β1, PGE2 and PAF) (Fadok, V A 1988, Cvetanovic, M 2004). This phenomenon of suppression of proinflammatory cytokine production by macrophages has been extended to include phagocytosis of apoptotic lymphocytes (Fadok, V A 2001).

In addition to macrophages, down regulation of pro-inflammatory cytokine release in response to apoptotic cells has also been demonstrated by non-phagocytizing cells including human fibroblasts, smooth muscle, vascular endothelial, neuronal and mammary epithelial cells (Fadok, V A 1988, 2000; McDonald, P P 1999, Cvetanovic M, 2006). Apoptotic neutrophils in contact with activated monocytes elicit an immunosuppressive cytokine response, with enhanced IL-10 and TGF-β production and only minimal TNF-α and IL-1β cytokine production (Byrne, A 2002). Byrne et al. concluded that the interaction between activated monocytes and apoptotic neutrophils may create a unique response, which changes an activated monocyte from being a promoter of the inflammatory cascade into a cell primed to deactivate itself and other cellular targets.

Techniques to identify and quantify apoptosis, and distinguish this event from necrosis, may include staining with nuclear stains allowing visualization of nuclear chromatin clumping (i.e. Hoeschst 33258 and acridine orange) (Earnshaw, W C 1995). Accurate identification of apoptosis is achieved with methods that specifically target the characteristic DNA cleavages. Agarose gel electrophoresis of extracted DNA fragments yields a characteristic ‘ladder’ pattern which can be used as a marker for apoptosis (Bortner, C D 1995). A lesser extent of DNA degradation produces hexameric structures called ‘rosettes’ where necrotic cells leave a nondescript smear (Pritchard, D M 1996). Terminal transferase deoxyuridine nick-end labeling of DNA break points (TUNEL analysis), which labels uridine residues of the nuclear DNA fragments, can also be used to quantify apoptosis (Gavrieli, Y 1992).

Several signature events in the process of apoptosis may also be quantified by flow cytometry. These include dissipation of the mitochondrial membrane potential which is an early apoptotic event, externalization of phosphotidylserine through capture with annexin V, loss of plasma membrane integrity and nuclear chromatin condensation (distinguishing live, apoptotic and necrotic cells), and activation of caspase enzymes (early stage feature of apoptosis) (Technical Bulletin—InVitrogen 2004).

Vascular endothelial cells, including human umbilical vein endothelial cells (HUVECs), are known to release potent vasodilators, including nitric oxide (NO) and prostacyclins. Treatment of HUVECs with ozonated serum, an oxidative stressor, results in a significant and steady increase in NO production. Moreover, during twenty-four (24) hour HUVEC incubation with ozonated serum, inhibition of E-selectin release (a proinflammatory mediator) and no effect on endothelin-1 production (a potent vasoconstrictor) has been reported (Valacchi, G 2000). Valacchi et al. has suggested that reinfusion of ozonated blood into patients, by enhancing release of NO, may induce vasodilation in ischemic areas and reduce hypoxia.

CRP is a product of inflammation the synthesis of which by the liver is stimulated by cytokines in response to an inflammatory stimulus. CRP activates the classic complement pathway and participates in the opsonization of ligands for phagocytosis. Initially suggested as solely a biomarker and powerful predictor of cardiovascular risk, CRP now appears to be a mediator of atherogenesis. CRP has a direct effect on promoting atherosclerotic processes and endothelial cell activation. CRP potently down regulates endothelial nitric oxide synthase (eNOS) transcription and destabilizes eNOS mRNA, which decreases both basal and stimulated nitric oxide (NO) release.

In a synchronous fashion, CRP has been shown to stimulate endothelin-1 (potent vasoconstrictor) and interleukin-6 release (proinflammatory cytokine), upregulate adhesion molecules, and stimulate monocyte chemotactic protein-1 while facilitating macrophage LDL uptake. More recently, CRP has been shown to facilitate endothelial cell apoptosis and inhibit angiogenesis, as well as potentially upregulate nuclear factor kappa-B, a key nuclear factor that facilitates the transcription of numerous pro-atherosclerotic genes. The direct pro-atherogenic effects of CRP extend beyond the endothelium to the vascular smooth muscle, where it directly upregulates angiotensin type 1 receptors and stimulates vascular smooth muscle migration, proliferation, neointimal formation and reactive oxygen species production. CRP has several deleterious effects (e.g., reduced survival, differentiation, function, apoptosis, and endothelial progenitor cell-eNOS mRNA expression) on endothelial progenitor cells which are important in neovascularization including induction of blood flow recovery in ischemic limbs and increase in myocardial viability after infarction.

Historically, ozone has been used as a disinfectant or sterilizing agent in a wide variety of applications. These include fluid-based technologies such as purification of potable water, sterilization of fluids in the semi-conductor industry, disinfection of wastewater and sewage and inactivation of pathogens in biological fluids. Ozone has also been used in the past as a topical medicinal treatment, as a systemic therapeutic and as a treatment of various fluids that were subsequently used to treat a variety of diseases. Specifically, there have been numerous attempts utilizing a variety of ozone-based technologies to treat an array of cardiovascular diseases in patients.

Previous technologies were incapable of measuring and differentiating between the amount of ozone that was delivered and the amount of ozone actually absorbed and utilized. This meant previous medicinal technologies for use in patients were incapable of measuring, reporting or differentiating the amount of ozone delivered from the amount that was actually absorbed and utilized. This problem made regulatory approval as a therapeutic unlikely. In the treatment of cardiovascular diseases, previous technologies were also incapable of measuring, reporting or differentiating the amount of ozone delivered from the amount that was actually absorbed by the fluid and utilized by the patient.

The inability to measure the amount of ozone absorbed may result in excessive absorption resulting in unacceptable levels of cellular necrosis in the leukocyte fraction of the treated blood, which when reinfused may result in promotion of an inflammatory response. Furthermore, any technology considered to treat cardiovascular disease utilizing blood ex vivo with ozone may have to be able to maintain the biological integrity of the fluid for its subsequent intended therapeutic use.

In addition, early approaches of mixing ozone with fluids employed gas-fluid contacting devices that were engineered with poor mass transfer efficiency of gas to fluids. Later, more efficient gas-fluid contacting devices were developed, but these devices used construction materials that were not ozone inert and therefore, reacted and absorbed ozone. This resulted in absorption of ozone by the construction materials making it impossible to determine the amount of ozone delivered to and absorbed by the fluid. Furthermore, ozone absorption by construction materials likely caused oxidation and the subsequent release of contaminants or deleterious byproducts of oxidation into the fluid.

Experimental research confirms the problem of ozone absorption by construction materials. An ozone/oxygen admixture at 1200 ppmv was passaged through a commercially available membrane oxygenator. For a period in excess of two hours, a majority of the ozone delivered to the device was absorbed by the construction materials. This data strongly suggests commercially available membrane gas-fluid contacting devices, made from ozone reactive materials, cannot be used with ozone, and supports the necessity to develop novel ozone-inert gas-fluid contacting devices.

In addition, prior methods do not quantify the amount of ozone that does not react with the biological fluid. The inability to measure residual-ozone has led to inaccurate and imprecise determinations of the amount of ozone actually absorbed and utilized by the fluid.

Prior technologies also include inefficient methods to mix ozone with fluids yielding irregular exposure. For example, relatively large amounts of ozone may be exposed to some of the fluid and less to other portions. The result of this inefficient mixing causes a wide variation in the amount of ozone exposed to the fluid. This wide variation in ozone exposure may cause diverse biochemical events including unacceptable levels of cellular necrosis in various portions of the fluid leading to untoward and irreproducible results.

Prior techniques also failed to recognize that fluids of varying composition display different absorption phenomena. The range of values for extracellular antioxidants in blood, including ascorbic acid (0.4-1.5 mg/dL), uric acid (2.1-8.5 mg/dL), bilirubin (0-1.0 mg/dL) and Vitamin A (30-65 μg/dL) and other oxidizable substrates, including cholesterol (140-240 mg/dL), LDL-cholesterol (100-159 mg/dL), HDL-cholesterol (33-83 mg/dL) and triglycerides (45-200 mg/dL), may alter the amount of ozone necessary to be delivered to the fluid, and subsequently absorbed and utilized to achieve a desired clinical effect.

### BRIEF SUMMARY OF THE INVENTION

In accordance with the present invention, methods for treatment of cardiovascular disease, and related physiological conditions, in a mammalian patient, including atherosclerosis, peripheral arterial occlusive disease, congestive heart failure, hypertension, cerebrovascular disease, dyslipidemia and vasospastic disorders, such as Raynaud's disease, are disclosed. The treatment provides a therapeutic approach which may affect reduction in edema, improvement in impaired blood flow, reduction in atherosclerotic plaques, regression in atherosclerotic plaque formation, relaxation of the vascular endothelium, reduction of inflammation, and reduction in lipids and lipid deposits.

The methods of the present invention involve subjecting an amount of blood, blood fractionate or other biological fluid extracorporeally to an amount of ozone delivered by an ozone delivery system resulting in the absorption of a quantifiable absorbed-dose of ozone, and re-infusing the treated fluid into the patient. The method may also provide for the maintenance of the biological integrity of the treated biological fluid.

The methods of the invention further include reinfusion of the treated fluid having the quantified absorbed dose of ozone into the mammalian subject to provide and elicit therapeutic effects which treat the disease, condition or symptoms of the disclosed diseases, as well as other diseases.

The methods of the present invention further provide for the manufacture of substances or compositions that are useful in the therapeutic treatment of cardiovascular disease and related symptoms and conditions of this disease. The methods of the present invention further provide for the use of such substances and compositions in the manufacture of medicaments or other administrable substances for the therapeutic treatment of cardiovascular disease and related symptoms and conditions of this disease.

The methods also involve subjecting an amount of blood, blood fractionate or other biological fluid extracorporeally to a measured amount of ozone such that the resulting absorption of a quantifiable absorbed-dose of ozone may result in a number of biochemical events including the induction of apoptosis in the leukocyte fraction. Reintroduction of the treated blood, blood fractionate or other biologic fluid may cause effects that include reduction of edema, improvement in impaired blood flow, reduction in atherosclerotic plaques, regression in atherosclerotic plaque formation, relaxation of the vascular endothelium, reduction of inflammation, and reduction in lipids and lipid deposits.

The methods may also result in the reduction in C-reactive protein (CRP) sufficient to elicit clinical benefit, which may include an anti-inflammatory response, neovascularization and vasodilation.

The methods of the present invention employ an ozone-delivery system for delivering and manufacturing a measured amount of an ozone/oxygen admixture, which is able to measure, control and report, and differentiate between, delivered-ozone and the absorbed-dose of ozone. The system may include gas-fluid contacting devices that maximize gas-fluid mass transfer. All gas contact surfaces of the system, including one or more gas-fluid contact devices and all gas-contacting pathways transporting ozone or an ozone/oxygen admixture into and away from the gas-fluid contacting device or devices, are made from ozone-inert construction materials that do not absorb ozone nor introduce contaminants or deleterious byproducts of oxidation into the fluid being treated.

The methods of treatment of cardiovascular disease involve, in certain embodiments, subjecting an amount of blood, blood fractionate or other biological fluid extracorporeally to a measured amount of ozone delivered by an ozone delivery system, resulting in the absorption of a quantifiable absorbed-dose of ozone and re-infusing the treated fluid into the patient to produce clinical benefits, including reduction in atherosclerotic plaques, regression in atherosclerotic plaque formation, reduction in triglycerides, cholesterol and other lipids, reduction in lipid deposits, relaxation of the vascular endothelium, reduction in edema and reduction of inflammation.

The results from the present methods of treatment of cardiovascular diseases may also include improved blood flow, reduction in episodes of intermittent claudication, reduction of the ischemic penumbra, reduction in blood pressure, reduction in extremity weakness and pain, improvement in extremity temperature and pallor and weight loss.

Diseases targeted as potential candidates for treatment by the methods disclosed in the present invention include atherosclerosis, peripheral arterial occlusive disease, cerebrovascular accident, angina pectoris and vasospastic disorders, such as Raynaud's disease, dyslipidemia, congestive heart failure and hypertension.

The methods of the present invention are directed to treating blood with ozone extracorporeally to generate leukocyte apoptosis, without excessive necrosis, sufficient to reduce edema, improve impaired blood flow, reduce atherosclerotic plaques, cause regression in atherosclerotic plaque formation, relax the vascular endothelium, reduce inflammation and reduce lipids and lipid deposits once the treated blood is reinfused.

The methods of the present invention are directed to treating blood with ozone extracorporeally is to cause, once the blood is reinfused to the patient, a reduction in CRP sufficient to elicit clinical benefit.

The methods of the present invention are directed to treatment of cardiovascular diseases, which may include atherosclerotic associated disorders, by delivery of a measured amount of ozone and subsequent absorption of a quantifiable absorbed-dose of ozone by blood or derivatives thereof extracorporeally, which may cause sufficient leukocyte apoptosis necessary to elicit clinical benefit when reinfused autologously into a patient. The methods are further directed to inducing sufficient leukocyte apoptosis without excessive necrosis to elicit clinical benefit when reinfused autologously into a patient.

The methods of the present invention are directed to treatment of cardiovascular diseases in a manner which causes sufficient leukocyte apoptosis, without excessive necrosis necessary, to elicit a reduction of inflammation when reinfused autologously into a patient.

The methods of the present invention are further directed to the treatment of cardiovascular diseases by methods which may elicit a reduction of inflammation when reinfused autologously into a patient by reducing proinflammatory cytokines (e.g. interferon-gamma, TNF-gamma, IL-6, IL-8 and IL-12) and/or an increase in anti-inflammatory cytokines (e.g. interleukin-4 and IL-10) released by immunomodulatory T cells.

The methods of the present invention are directed to eliciting a reduction of inflammation when treated fluids, such as blood, are reinfused autologously into a patient, resulting in a number of clinical benefits including improvement in blood flow yielding enhanced oxygenation.

The methods of the present invention are directed to providing treatment of cardiovascular diseases by delivery of a measured amount of ozone to, and subsequent absorption of a quantifiable absorbed-dose of ozone by, blood or derivatives thereof extracorporeally, which may cause sufficient leukocyte apoptosis without excessive necrosis necessary to elicit a reduction of edema when reinfused autologously into a patient and/or to cause a reduction of CRP when reinfused autologously into a patient to elicit clinical benefit including an anti-inflammatory response. Other clinical benefits of the methods of treatment include an increase in blood flow to ischemic tissue. Increased blood flow to ischemic tissue may be evaluation by a variety of diagnostic tools including MRI, CT perfusion and Doppler imaging techniques.

The methods of the present invention are directed to providing a treatment of cardiovascular diseases, which may include atherosclerotic associated disorders in a mammalian patient, by treating blood by a discontinuous flow method, the method comprising connecting a subject to a device for withdrawing blood, withdrawing blood and delivering a measured amount of ozone to the blood under conditions which may maintain the biological integrity of the blood. The treated blood is subsequently re-infused into the subject.

The methods of the present invention are directed to providing a treatment of cardiovascular diseases, the method comprising connecting a subject to a device for withdrawing blood, withdrawing blood containing blood cells from the subject, separating a non-cellular fraction from the blood and delivering a measured amount of ozone to the fraction, under conditions which may maintain the biological integrity of the blood fraction. The treated fraction is subsequently recombined with the blood cells and re-infused into the subject.

The methods of the present invention are directed to providing a treatment for the reduction of cholesterol, triglycerides and other lipids in a mammalian patient by treating blood by a discontinuous flow method. The method comprising connecting a subject to a device for withdrawing blood, withdrawing blood and delivering a measured amount of ozone to the blood under conditions which may maintain the biological integrity of the blood. The treated blood is subsequently re-infused into the subject.

The methods of the present invention are further directed to providing for the reduction of cholesterol, triglycerides and other lipids in a mammalian patient by treating blood, or a fraction thereof, including plasma or serum, by a discontinuous flow method, said method comprising connecting a subject to a device for withdrawing blood, withdrawing blood containing blood cells from the subject, separating a non-cellular fraction from the blood and delivering a measured amount of ozone to the fraction with ozone, under conditions which may maintain the biological integrity of the blood fraction. The treated fraction is recombined with the blood cells and subsequently re-infused into the subject.

The methods of the present invention are directed to providing treatments that cause a rapid regression of coronary atherosclerosis in a patient, and/or a reduction in atherosclerotic plaques in a patient, and/or that reverse the progressive luminal narrowing of an artery due to expansion of a fibrous plaque during artherosclerotic plaque development.

The methods of the present invention are also directed to treatments that inhibit rupture of an atherosclerotic plaque, and/or that inhibit denudation of the vascular endothelium in patients suffering from atherosclerosis.

The methods of the present invention are directed to providing a treatment that causes a rapid regression of coronary atherosclerosis in a patient. However, retardation in progression and regression of atherosclerotic plaques may not necessarily be accompanied by a significant reduction in serum lipid levels. As discussed, atherosclerosis has a significant immune-modulated inflammatory component. Therefore, the ability of the method to prevent and treat atherosclerosis may be at least partially due to its anti-inflammatory action which causes the secretion of anti-inflammatory cytokines, thereby reducing an autoimmune response.

The methods of the present invention are directed to providing a treatment for hyperlipidemia by effecting a reduction in triglycerides, cholesterol and other lipids. The methods of the present invention are directed to providing therapeutic treatments for weight loss secondary to a reduction or enhanced clearance of triglycerides.

The methods of the present invention are further directed to providing a treatment to reduce lipomatous masses and other lipid deposits.

The methods of the present invention are directed to providing therapeutic treatments to reduce the frequency of episodes of angina pectoris secondary to atherosclerosis, and to inhibit or prevent the formation of an atheroembolism in patients with advanced or diffuse atherosclerosis. The methods may also be directed to treatments that promote or cause the clearance of a cholesterol embolism.

The methods of the present invention are directed to providing therapeutic treatments where improvement in the blood rheology of an atherosclerotic patient with impaired blood circulation is achieved.

The methods of the present invention are directed to providing therapeutic treatments that reduce the frequency of intermittent claudication in patients with peripheral arterial occlusive disease.

The methods of the present invention are directed to providing therapeutic treatments that promote the repair of non-healing ulcerations in patients with peripheral arterial occlusive disease.

The methods of the present invention are further directed to providing therapeutic treatments for visceral ischemia and/or mesenteric angina in patients with atherosclerosis.

The methods of the present invention are also directed to providing therapeutic treatments for patients that have been diagnosed with abdominal aortic aneurysm.

### BRIEF DESCRIPTION OF DRAWING

To further clarify the present invention, treatment systems of the present invention using an ozone delivery system are illustrated in the appended drawing, which schematically illustrate what is currently considered the best mode for carrying out the invention;

FIG. 1 illustrates, in a schematic diagram, alternate methods of carrying out treatment of a fluid from a patient, comprising a continuous loop format and, alternatively, a discontinuous flow method.

### DETAILED DESCRIPTION OF THE EMBODIMENTS OF THE INVENTION

### Definitions

As used herein, “comprising,” “including,” “containing,” “characterized by,” and grammatical equivalents thereof are inclusive or open-ended terms that do not exclude additional, unrecited elements or method steps, but also include the more restrictive terms “consisting of and “consisting essentially of.”

As used herein, and in the appended claims, the singular forms, for example, “a”, “an”, and “the,” include the plural, unless the context clearly dictates otherwise. For example, reference to “a gas-fluid contacting device” includes a plurality of such gas-fluid contacting devices, and reference, for example, to a “protein” is a reference to a plurality of similar proteins, and equivalents thereof.

An “ozone/oxygen admixture” refers to a concentration of ozone in an oxygen carrier gas. Various units of concentration utilized by those skilled in the art include micrograms of ozone per milliliter of oxygen, parts (ozone) per million (oxygen) by weight (‘ppm’) and parts per million by volume (‘ppmv’). As a unit of concentration for ozone in oxygen, ppmv is defined as the molar ratio between ozone and oxygen. One ppmv ozone is equal to 0.00214 micrograms of ozone per milliliter of oxygen. Additionally, one ppm ozone equals 0.00143 micrograms of ozone per milliliter of oxygen. In terms of percentage ozone by weight, 1% ozone equals 14.3 micrograms of ozone per milliliter of oxygen. All units of concentration and their equivalents are calculated at standard temperature and pressure (i.e. 25° C. at 1 atmosphere).

“Delivered-ozone” is the amount of ozone contained within a volume of an ozone/oxygen admixture that is delivered to a fluid, and is synonymous with the delivery of a measured amount of ozone.

“Absorbed-ozone” is the amount of delivered-ozone that is actually absorbed and utilized by an amount of fluid, and is synonymous with a quantifiable absorbed dose of ozone.

“Residual-ozone” is the amount of delivered-ozone that is not absorbed such that:

Residual-ozone=delivered ozone−absorbed-dose of ozone

An “interface” is defined as the contact between a fluid and an ozone/oxygen admixture.

“Interface-time” is defined as the time that a fluid resides within a gas-fluid contacting device and is interfaced with an ozone/oxygen admixture.

“Interface surface area” is defined as the dimensions of the surface within a gas-fluid contacting device over which a fluid flows and contacts an ozone/oxygen admixture.

“Elapsed-time” is the time that a fluid circulates throughout an ozone delivery system, including passage through one or more gas-fluid contacting devices, connecting tubing and an optional reservoir.

“Ozone-inert materials” are defined as construction materials that do not react with ozone in a manner that introduces contaminants or deleterious byproducts of oxidation of the construction materials into a fluid, and materials that do not absorb ozone.

“Non-reactive” is defined as not readily interacting with other elements or compounds to form new chemical compounds.

“Measured-data” is defined as information collected from various measuring components (such as an inlet ozone concentration monitor, exit ozone concentration monitor, gas flow meter, fluid pump, data acquisition device, humidity sensor, temperature sensor, pressure sensor, absorbed oxygen sensor) throughout the system.

“Calculated-data” is defined as the mathematical treatment of measured-data by a data acquisition device.

“Absorption of ozone by a biological fluid” is defined as the phenomenon wherein ozone reacts with the fluid being treated by a variety of mechanisms, including oxidation. Regardless of the mechanism involved, the reaction occurs instantaneously, and the products of this reaction include oxidative products, of which lipid peroxides are an example.

A “biological fluid” is defined as a composition originating from a biological organism of any type. Examples of biological fluids include blood, blood products and other fluids, such as saliva, urine, feces, semen, milk, tissue, tissue samples, homogenized tissue samples, gelatin and any other substance having its origin in a biological organism. Biological fluids may also include synthetic materials incorporating a substance having its origin in a biological organism, such as a vaccine preparation containing alum and a virus (the virus being the substance having its origin in a biological organism), cell culture media, cell cultures, viral cultures, and other cultures derived from a biological organism.

A “blood product” is defined as including blood fractionates and therapeutic protein compositions containing proteins derived from blood. Fluids containing biologically active proteins other than those derived from blood may also be treated by the method.

“In vivo” use of a material or compound is defined as the introduction of a material or compound into a living human, mammal or vertebrate.

“In vitro” use of a material or compound is defined as the use of the material or compound outside a living human, mammal or vertebrate, where neither the material nor compound is intended for reintroduction into a living human, mammal or vertebrate. An example of an in vitro use would be the analysis of a component of a blood sample using laboratory equipment.

“Ex vivo” use of a process is defined as using a process for treatment of a biological material such as a blood product outside of a living human, mammal, or vertebrate. For example, removing blood from a human and subjecting that blood to a method to treat a cardiovascular disease is defined as an ex vivo use of that method if the blood is intended for reintroduction into that human or another human. Reintroduction of the human blood into that human or another human would be an in vivo use of the blood, as opposed to an ex vivo use of the method.

“Extracorporeal” is defined as a state wherein blood or blood fractionate is treated outside (ex vivo) of the body, for example, in the delivery of a measured amount of ozone to a sample of patient's blood.

“Synthetic media” is defined as an aqueous synthetic blood or blood product storage media.

A “pharmaceutically-acceptable carrier” or “pharmaceutically-acceptable vehicle” is defined as any liquid including water, saline, a gel, salve, solvent, diluent, fluid ointment base, liposome, micelle or giant micelle, which is suitable for use in contact with a living animal or human tissue without causing adverse physiological responses, and which does not interact with the other components of the composition in a deleterious manner.

“Biologically active” is defined as capable of effecting a change in the living organism or component thereof.

The “biological integrity of a biological fluid” is a quality or state of a fluid that, subsequent to the method of treating for cardiovascular diseases described herein, sufficiently maintains its functionality upon re-infusion into a mammalian patient.

“Cardiovascular diseases” are defined as those diseases which may include atherosclerosis, arteriosclerosis, peripheral arterial occlusive disease, cerebrovascular disease, hypertension, Raynaud's disease, dyslipidemia and congestive heart failure.

“Edema” is defined as a condition of abnormally large fluid volume in the circulatory system or in tissues between the body's cells (interstitial spaces).

“C-reactive protein” is defined as a liver-synthesized, acute phase reactant protein regarded as a marker of acute inflammation capable of activating the classical compliment pathway and opsonizing ligands for phagocytosis.

The present invention provides methods for therapeutic treatment of cardiovascular diseases mediated by the delivery of a measured amount of ozone to a sample of a patient's blood, blood fractionate or other fluid, extracorporeally, through the use of an ozone delivery system. A quantifiable absorbed-dose of ozone absorbed by the fluid is subsequently re-infused into the same patient. This autologous blood sample which contains a quantifiable absorbed-dose of ozone may affect reduction in edema, improvement in impaired blood flow, reduction in atherosclerotic plaques, regression in atherosclerotic plaque formation, reduction of the ischemic penumbra, relaxation of the vascular endothelium, reduction of inflammation, and reduction in lipids and lipid deposits. The method may therefore be useful in the treatment of cardiovascular diseases including atherosclerosis, peripheral arterial occlusive disease, congestive heart failure, hypertension, cerebrovascular disease, dyslipidemia and vasospastic disorders, including Raynaud's disease, in a mammalian patient. Positive treatment outcomes may be measured by various methods of quantification or qualification, and include a measurable reduction in cholesterol, triglycerides and other lipids, reduction in the frequency, severity and duration of episodes of intermittent claudication, reduction in extremity numbness, weakness and loss of movement, improvement in extremity pallor, temperature and pulse, reduction in elevated blood pressure, and weight loss.

Methods of the present invention are directed to providing therapeutic treatment of cardiovascular diseases, including a method of delivering a measured amount of ozone to, and subsequent absorption of a quantifiable absorbed-dose of ozone by, blood or derivatives thereof extracorporeally, which may cause sufficient leukocyte apoptosis without excessive necrosis necessary to elicit clinical benefit when reinfused autologously into a patient.

The present invention also provides methods for delivery of a measured amount of ozone and subsequent absorption of a quantifiable absorbed-dose of ozone by blood or derivatives thereof, extracorporeally, where, following reinfusion autologously into a patient, may cause a reduction in CRP sufficient to elicit clinical benefit. The disclosed methods may also affect relaxation of vascular endothelium, and may involve release of vasodilators, including nitric oxide and prostacyclins sufficient to elicit clinical benefit.

In one embodiment of the invention, methods are provided for treatment of blood, blood fractionate or other fluid, and use of this treated blood, blood fractionate or other fluid in the treatment of cardiovascular diseases which may include atherosclerotic and dysfunctional endothelial associated disorders, in a mammalian patient by administration to the patient of such treated blood, blood fractionate or other fluid.

These methods may comprise extracorporeally subjecting an aliquot of a mammalian patient's blood, or the separated cellular fractions of the blood, or mixtures of the separated cells, including platelets, to a measured amount of ozone such that the treated fluid absorbs a quantifiable absorbed-dose of ozone. On reintroduction of this autologous aliquot to the patient's body, the blood, blood fractionate or other fluid with a quantifiable absorbed-dose of ozone provides certain beneficial effects. These effects may result in the reduction and/or inhibition of atherosclerotic plaque formation, deposition or plaque rupture, and stimulation of the activity of a functionally deficient endothelium.

The effects of blood, blood fractionate or other fluid that has absorbed a quantifiable absorbed-dose of ozone, when reinfused into a mammalian patient's body, may include changes in lipid metabolism and enhancement of the immune system through stimulation of leukocytes (i.e. cell-cell interaction or cytokine release) throughout the peripheral blood of the patient. This may lead to a reduction in atherosclerotic plaque formation and deposition, a reduction in lipids and lipid deposits, a relaxation of the vascular endothelium, reduction in edema, and reduction of inflammation. These effects may result in a reversal in progressive luminal narrowing in arteries, a reduction in the rupture or denudation of plaques, and an increase in vasodilation (i.e. promotion of vasodilators or inhibition of vasoconstrictors), thereby decreasing the incidence of atheroembolism (cholesterol embolism), improving endothelial function including endothelial cellular repair or replacement, and improving blood flow yielding enhanced oxygenation.

Clinical signs of these effects may include reductions in episodes and severity of intermittent claudication, reduction of the ischemic penumbra, a reduction in elevated blood pressure, reduction in extremity weakness and pain, normalization of extremity temperature and an improvement in pallor. In addition, weight loss may be a result of this treatment approach.

Regarding disorders involving atherosclerotic plaque formation, deposition and plaque rupture, the present methods provide for therapeutic treatment and prophylaxis of a wide variety of such mammalian disorders, including cardiovascular diseases, such as atherosclerosis, peripheral arterial occlusive disease, cerebrovascular disease (stroke and transient ischemic attack), myocardial infarction, angina, hypertension, retinal ischemia, renal failure, abdominal aortic aneurysm, and hyperlipidemia.

For those disorders involving endothelial dysfunction, the present methods provide for therapeutic treatment and prophylaxis of a wide variety of such mammalian disorders including cardiovascular diseases, such as atherosclerosis, peripheral arterial occlusive disease, congestive heart failure, cerebrovascular disease (stroke), myocardial infarction, angina, hypertension, vasospastic disorders such as Raynaud's disease, cardiac syndrome X, and migraine.

The therapeutic effect of blood, or a derivative thereof, which has absorbed a quantifiable absorbed-dose of ozone, may be the induction of sufficient leukocyte apoptosis without excessive necrosis necessary to elicit an anti-inflammatory response when reinfused autologously into a patient. The induction of apoptosis without excessive necrosis in the leukocyte fraction of the blood that has been treated may be evaluated by a number of diagnostic methods including light microscopy with nuclear stains, electrophoretic analysis of DNA fragmentation, TUNEL analysis and multiparameter flow cytometry.

An effect of blood, or blood derivative thereof, which has absorbed a quantifiable absorbed-dose of ozone, may result in the reduction of CRP when reinfused autologously into a patient and may elicit clinical benefit, including an anti-inflammatory response, neovascularization and vasodilation.

The effects of blood, blood fractionate or other fluid which has absorbed a quantifiable absorbed-dose of ozone, when reinfused into a mammalian patient's body, may include effects that reduce edema.

In addition, the effects of blood, blood fractionate or other fluid which has absorbed a quantifiable absorbed-dose of ozone, when re-infused into a mammalian patient's body may include effects that increase blood flow to ischemic tissue. This reduction can be evaluated by a variety of diagnostic tools including MRI and Doppler imaging techniques.

Furthermore, the effects of blood, blood fractionate or other fluid which has absorbed a quantifiable absorbed-dose of ozone, when re-infused into a mammalian patient's body, may include effects that include reduction of inflammation. Reduction of inflammation may occur though a reduction in pro-inflammatory cytokines (e.g. interferon-gamma, TNF-gamma, IL-6, IL-8 and IL-12) and/or an increase in anti-inflammatory cytokines (e.g. interleukin-4 and IL-10) released by immunomodulatory T cells. The effect of reducing inflammation may result in any number of clinical benefits including improvement in blood flow yielding enhanced oxygenation.

The effect of treated blood or blood derivative thereof with ozone by the present method to induce apoptotic leukocytes without excessive necrosis, when re-infused into a mammalian patient's body may include effects that include reduction of inflammation. Reduction of inflammation may occur though a reduction in pro-inflammatory cytokines (e.g. interferon-gamma, TNF-gamma, IL-6, IL-8 and IL-12) and/or an increase in anti-inflammatory cytokines (e.g. interleukin-4 and IL-10) released by immunomodulatory cells. The effect of reducing inflammation may result in any number of clinical benefits in the treatment of cardiovascular diseases, including improvement in blood flow yielding enhanced oxygenation.

In accordance with the methods of the present invention, reintroduction of treated blood, blood fractionate or other fluid autologously to a mammalian patient may be accomplished through a variety of routes, including intravenous, intramuscular and subcutaneous routes, or any combination thereof.

An ozone delivery system utilized in the treatment of cardiovascular diseases delivers a measured amount of an ozone/oxygen admixture and is able to measure, control, report and differentiate between the delivered-ozone and absorbed-dose of ozone. The system provides a controllable, measurable, accurate and reproducible amount of ozone that is delivered to a controllable, measurable, accurate and reproducible amount of a biological fluid, and controls the rate of ozone absorption by the fluid resulting in a quantifiable absorbed-dose of ozone used in the treatment of cardiovascular diseases. The system may accomplish this by using a manufacturing component, control components, measuring components, a reporting component and calculating component (such as an ozone generator, gas flow meter, fluid pump, variable pitch platform, data acquisition device, inlet ozone concentration monitor, and exit ozone concentration monitor) that cooperate to manufacture and deliver a measured, controlled, accurate and reproducible amount of ozone, i.e., the delivered-ozone, to a fluid through the use of one or more gas-fluid contacting devices that provides for the interface between the ozone/oxygen admixture and fluid. Using control components, measuring components, a reporting component and calculating component (such as a gas flow meter, fluid pump, variable pitch platform, data acquisition device, inlet ozone concentration monitor and exit ozone concentration monitor) that cooperate, the system may instantly differentiate the delivered-ozone from the absorbed-dose of ozone.

The system utilizes (a gas flow meter, fluid pump, variable pitch platform, data acquisition device, inlet ozone concentration monitor, and exit ozone concentration monitor) control components, measuring components, a reporting component and calculating component that cooperate and instantly report data that may include the delivered-ozone, residual-ozone, absorbed-dose of ozone, interface-time, elapsed-time and the amount and flow rate of the fluid delivered to the gas-contacting device.

A particularly suitable ozone delivery system that may be used in carrying out the methods of the present invention is disclosed in U.S. Pat. No. 7,736,494 and co-pending application Ser. No. 12/813,371, the contents of which are incorporated herein in their entirety. The disclosed ozone delivery system is particularly and uniquely constructed such that all ozone-contacting surfaces of the device are made of ozone-inert material so that the amount of ozone that is actually absorbed by the biological fluid being treated is accurately determinable. That is, by virtue of being constructed with ozone-inert materials in all ozone-contacting surfaces, no ozone is absorbed by the device itself, and the determination of the amount of ozone absorbed by the biological fluid is not inaccurately reflected as a result of ozone being absorbed by any structure of the device

The ozone delivery system utilizes measuring components, reporting components and calculating components (such as an inlet ozone concentration monitor, exit ozone concentration monitor, gas flow meter, fluid pump, data acquisition device) that cooperate together to determine certain calculated-data including the delivered-ozone, the residual-ozone and the absorbed-dose of ozone.

Delivered-ozone is an amount of ozone calculated by multiplying the measured volume of ozone/oxygen admixtures, as reported by gas flow meters, by the measured concentration of ozone within the ozone/oxygen admixture as it enters the gas-fluid contacting device, as reported by the inlet ozone concentration monitor. The measured volume of ozone/oxygen admixtures is calculated by multiplying the measured gas flow reported by gas flow meters, by the elapsed-time.

Residual-ozone is an amount of ozone calculated by multiplying the measured volume of ozone/oxygen admixtures, as reported by gas flow meters, by the measured concentration of ozone within the ozone/oxygen admixture exiting the gas-fluid contacting device, as reported by the exit ozone concentration monitor. The measured volume of ozone/oxygen admixtures is calculated by multiplying the measured gas flow reported by gas flow meters, by the elapsed-time.

The quantifiable absorbed-dose of ozone is an amount of ozone calculated by subtracting the amount of residual-ozone from the amount of delivered-ozone. The quantifiable absorbed-dose of ozone in the methods of the invention may range from 1 to 10,000,000 micrograms per milliliter of fluid, and may be between 1 and 10,000 ug per milliliter of fluid.

All measured-data, including measured data from the gas flow meters, inlet and exit ozone concentration monitors, the fluid pump, temperature sensors, pressure sensors, absorbed oxygen sensor and humidity sensors are reported to a data acquisition device. The data acquisition device has instant, real-time reporting, calculating and data storing capabilities to process all measured data. The data acquisition device may use any measured data or any combination of measured data as variables to produce calculated-data. Examples of calculated-data may include delivered-ozone, residual-ozone, absorbed-dose of ozone, absorbed-dose of ozone per unit volume of fluid, and the quantifiable absorbed-dose of ozone per unit volume of fluid per unit time.

An ozone delivery system particularly suitable to the present invention includes an ozone generator for the manufacture and control of a measured amount of an ozone/oxygen admixture and where the admixture volume contains the delivered-ozone. A commercially available ozone generator capable of producing ozone in a concentration range between 10 and 3,000,000 ppmv of ozone in an ozone/oxygen admixture may be employed. Ozone/oxygen admixture concentrations entering the gas-fluid contacting device are instantly and constantly measured in real time, through an inlet ozone concentration monitor that may utilize UV absorption as a detection methodology. A flow meter controls and measures the delivery of the delivered-ozone in an ozone/oxygen admixture to the gas-fluid contacting device at a specified admixture flow rate. Ozone/oxygen admixture flow rates are typically in the range between 0.1 and 5.0 liters per minute.

Measurement of the humidity of the ozone/oxygen admixture delivered to the gas-fluid contacting device may be included through the use of a humidity sensor. A humidity sensor port may be provided in the ozone/oxygen admixture connecting tubing; however, it can be placed in a variety of locations. For example, the humidity sensor may be located in the connecting tubing prior to the admixture's entrance into gas-fluid contacting device.

Measurement of the temperature within the gas-fluid contacting device during the interface-time may be provided by inclusion of a temperature sensor port in the gas fluid contacting device through which a temperature sensor may be inserted. The temperature at which ozone/oxygen admixtures interface fluids ranges from 4° C. to 100° C., and may be performed at ambient temperature, 25° C., for example. The temperature at which the interface occurs can be controlled by placing the gas-fluid contacting device, optional reservoir, and both gas and fluid connecting tubing in a temperature controlled environment and/or by the addition of heating or cooling elements to the gas-fluid contact device.

Measurement of the pressure within the gas-fluid contacting device during the interface-time is provided by inclusion of a pressure sensor port in the gas-fluid contacting device through which a pressure sensor may be inserted. The pressure at which an ozone/oxygen admixtures interfaces with a fluid ranges from ambient pressure to 50 psi and may be performed between ambient pressure and 3 psi, for example. A pressure sensor port may be provided in each gas-fluid contacting device to measure and report the pressure at which the interface occurs.

The concentration of the ozone/oxygen admixtures exiting the gas-fluid contacting device, and where the admixture volume contains the residual-ozone, are instantly and constantly measured in real time through an exit ozone concentration monitor that may utilize UV absorption as a detection methodology.

A fluid pump controls and measures the flow rate of the fluid delivered to the gas-fluid-contacting device at a specified fluid flow rate. Fluid flow rates through the gas-fluid contacting device typically will range from 1 ml to 100 liters per minute, and for example, may be between 1 ml to 10 liters per minute. The fluid is generally contained within a closed-loop design and may be circulated through the gas-fluid contacting device once or multiple times.

Measurement of the amount of oxygen absorbed into a fluid while it interfaces with the ozone/oxygen admixture within the gas-fluid contacting device may be provided through the use of an absorbed oxygen sensor. The sensor is inserted within the absorbed oxygen sensor port located in the tubing as it exits the gas-fluid contacting device. Measurement of absorbed oxygen may be recorded in various units, including ppm, milligrams/liter or percent saturation.

The ozone delivery system may also include a fluid access port for fluid removal. The port is generally located in the tubing member after the fluid exits through the fluid exit port of the gas-fluid contacting device and prior to an optional reservoir.

A data acquisition device, such as a DAQSTATION (Yokogawa), for example, reports, stores and monitors data instantly and in real-time, and performs various calculations and statistical operations on data acquired. Data is transmitted to the data acquisition device through data cables, including data from ozone concentration monitors, flow meters, a humidity sensor, temperature sensors, pressure sensors, a fluid pump and an absorbed oxygen sensor.

Calculated-data in carrying out the methods of the present invention include delivered-ozone, residual-ozone, and the quantifiable absorbed-dose of ozone. Measurement of the volume of the ozone/oxygen admixture delivered can be calculated though data provided from the flow meter and the time measurement capability of the data acquisition device. Measurement of the volume of fluid delivered to the gas-fluid contacting device can be calculated by the data acquisition device utilizing fluid flow rate data transmitted from the fluid pump.

The elapsed-time can be measured and controlled through the data acquisition device. The elapsed-time that the fluid circulates through the apparatus, including the gas-fluid contacting device, and is interfaced with an ozone/oxygen admixture can vary, generally for a duration of up to 120 hours. The interface-time may also be measured by the time measuring capacity of the data acquisition device. The interface-time between a fluid and an ozone/oxygen admixture may be controlled through a composite of controls. These controls include the angle of the gas fluid contacting device, the fluid flow rate via the fluid pump, and the time controlling capacity of the data acquisition device. The interface-time may vary in duration of up to 720 minutes, and generally within duration of up to 120 minutes.

Controllable variables for an ozone delivery system may include delivered amounts and concentrations of ozone in the entering ozone/oxygen admixtures, fluid flow rates, admixture flow rates, temperature in the gas-fluid contacting device, interface-time between fluid and admixture, and the elapsed-time that the fluid may circulate through the apparatus and interface with an ozone/oxygen admixture.

Measurable variables may include ozone/oxygen admixture flow rates, amounts and concentrations of ozone in the entrance and exit ozone/oxygen admixtures, fluid flow rates, temperature and pressure in the gas-contacting device, humidity of the entrance admixture to the gas-fluid contacting device, absorbed oxygen by the fluid, interface-time and elapsed-time.

Data representing controllable variables and measurable variables acquired by the apparatus allows for a variety of calculations including delivered-ozone, residual-ozone, quantifiable absorbed-dose of ozone, quantifiable absorbed-dose of ozone per unit volume of fluid and the quantifiable absorbed-dose of ozone per unit volume of fluid per unit time.

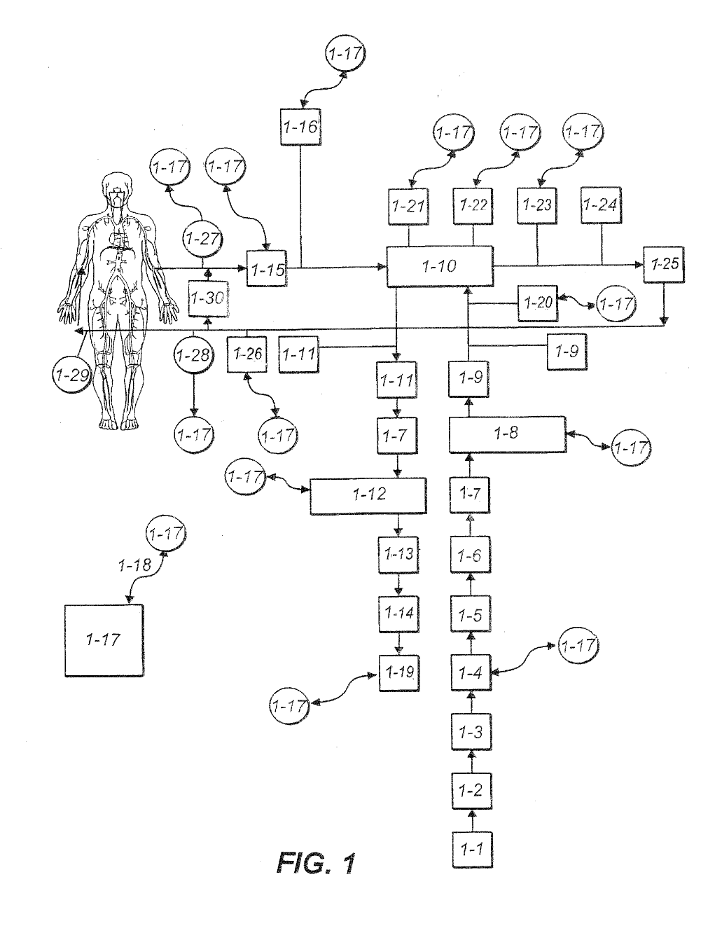

FIG. 1 schematically illustrates an embodiment of the present invention where fluid that has been taken from a subject is extracorporeally interfaced with an ozone/oxygen admixture. In general, blood may be circulated in a discontinuous manner where a fluid (e.g., an aliquot of blood) has been removed from a patient and is introduced into an ozone delivery system through a common reservoir, and is recirculated in a closed loop format. Alternatively, fluid may be circulated in a continuous loop format in a venovenous extracorporeal exchange format. As an example, this continuous loop can be established through venous access of the antecubital veins of both right and left arms. Prior to establishing a discontinuous closed loop format, blood from the patient may be anticoagulated with citrate or any other suitable anticoagulant before being introduced in to the reservoir. For an extracorporeal continuous loop circuit, a patient may optionally be anticoagulated with heparin or any other suitable anticoagulant known to those skilled in the art.

For the gas flow in either the discontinuous format or continuous loop system, oxygen flows from a pressurized cylinder (1-1), through a regulator (1-2), through a particle filter (1-3) to remove particulates, through a flow meter (1-4) where the oxygen and subsequent ozone/oxygen admixture flow rate is controlled and measured. The oxygen proceeds through a pressure release valve (1-5), through an ozone generator (1-6) where the concentration of the ozone/oxygen admixture is manufactured and controlled and where the admixture volume includes the delivered-ozone. The ozone/oxygen admixture flows through an optional moisture trap (1-7), to reduce moisture.

The admixture proceeds through an inlet ozone concentration monitor (1-8) that measures and reports the inlet ozone concentration of the ozone/oxygen admixture that contains the delivered-ozone. This real-time measurement may be based on ozone's UV absorption characteristics as a detection methodology. The ozone/oxygen admixture then passes through a set of valves (1-9) used to isolate a gas-fluid contacting device for purging of gasses. The ozone/oxygen admixture may pass an optional humidity sensor (1-20) where humidity may be measured and recorded, and into a gas-fluid contacting device (1-10) where it interfaces with fluid. The interface-time between fluid and ozone/oxygen admixture may be controlled through adjustment of a variable pitch platform, a fluid pump and the time controlling capacity of the data acquisition device.

The interface-time may then be measured by the data acquisition device (1-17). Temperature (1-21) and pressure (1-22) may be measured by the use of optional temperature and pressure sensors, respectively, inserted into their respective ports. The resultant ozone/oxygen admixture containing the residual-ozone exits the gas-fluid contacting device and flows through the exit purge valves (1-11), through a moisture trap (1-7), through an exit ozone concentration monitor (1-12), which may utilize a similar detection methodology as the inlet ozone concentration monitor (1-8), that measures and reports the exit ozone/oxygen admixture concentration. The exiting ozone/oxygen admixture then proceeds through a gas drier (1-13), through an ozone destructor (1-14) and a flow meter (1-19).

In the fluid flow for the discontinuous format, blood is introduced into the reservoir (1-30). In the continuous loop system, intravenous blood flows from the patient through tubing through a pressure gauge (1-27) which monitors the pressure of the blood flow exiting the patient. Generally, the pressure of the blood exiting the patient ranges from a negative pressure of 100-200 mm Hg, and may be between a negative pressure of 150 and 200 mm Hg, with a maximum cutoff pressure of minus 250 mm Hg. In either format, the blood flows through a fluid pump (1-15) and is optionally admixed with heparin or other suitable anticoagulant as provided by an optional heparin pump (1-16).

The blood then passes through the gas-fluid contacting device (1-10) where it interfaces with the ozone/oxygen admixture containing the delivered-ozone. Ports for the insertion of sensors may be located in the gas-fluid contacting device for the measurement of temperature and pressure, respectively. After interfacing with the ozone/oxygen admixture, the fluid exits into tubing that may contain a port for an optional absorbed oxygen sensor (1-23) followed by a fluid access port (1-24). The blood continues through an air/emboli trap (1-25) that removes any gaseous bubbles or emboli, and the blood then continues through a fluid pump (1-26).

In a discontinuous format, the blood is then directed back into the reservoir (1-30) any may continue in a recirculating mode, passaging as often as required. In the continuous loop format, the blood is directed into a pressure gauge (1-28) which monitors the pressure of the blood flow before returning the fluid to the patient. Generally, the pressure of the blood entering the patient ranges from a pressure of 100-200 mm Hg, and may be between 150 and 200 mm Hg, with a maximum cutoff pressure of 250 mm Hg. The blood continues through a priming fluid access port (1-29) that allows for the removal of the priming fluid from the extracorporeal loop. The blood is then re-infused directly into the patient.

A data acquisition device (1-17), such as a DAQSTATION (Yokogawa), for example, has time measurement capabilities, reports, stores and monitors data instantly and in real-time, and performs various calculations and statistical operations on data acquired. All data is transmitted to the data acquisition device through data cables (1-18), including: data from ozone concentration monitors (1-8) and (1-12), flow meters (1-4) and (1-19), humidity sensor (1-20), temperature sensor (1-21), pressure sensor (1-22), fluid pumps (1-15) and (1-26), pressure gauges (1-27) and (1-28), and absorbed oxygen sensor (1-23). The elapsed time, a composite of both the interface time and the period of time that the fluid circulates through the other elements of the apparatus can be measured and controlled through the data acquisition device (1-17).

Other possible configurations for an extracorporeal blood circuit known to those skilled in the art are included within the spirit of this disclosure.

One or more gas-fluid contacting devices may be included in an ozone delivery system to increase the surface area of a fluid to be treated allowing for an increase in the mass transfer efficiency of the ozone/oxygen admixture. Gas-fluid contacting devices may encompass the following properties: closed and isolated from the ambient atmosphere, gas inlet and outlet ports for the entry and exit of ozone/oxygen admixtures, fluid inlet and outlet ports for the entry and exit of a fluid, components (temperature sensor, pressure sensor and data acquisition device) for the measurement and reporting of temperature and pressure within a gas-fluid contacting device, generation of a thin film of the fluid as it flows within a gas-fluid contacting device and construction from ozone-inert construction materials including, quartz, ceramic composite, borosilicate, stainless steel, PFA and PTFE.

Gas-fluid contacting devices include designs that encompass surfaces that may be horizontal or approaching a horizontal orientation. These surfaces may include ridges, indentations, undulations, etched surfaces or any other design that results in a contour change and furthermore, may include any pattern, regular or irregular, that may disrupt the flow, disperse the flow or cause turbulence. These surfaces may or may not contain holes through which a fluid passes through. The surface of the structural elements may have the same or different pitches. Designs of gas-fluid contacting devices may include those that involve one or more of the same shaped surfaces or any combination of different surfaces, assembled in any combination of ways to be encompassed within the device which may include cones, rods, tubes, flat and semi-flat surfaces, discs and spheres.

The interface between an ozone/oxygen admixture and a fluid may be accomplished by the use of a gas-fluid contact device that generates a thin film of the fluid that interfaces with the ozone-oxygen admixture as it flows through the device. One of skill in the art will appreciate that generation of any interface that increases the surface area of the fluid and thereby maximizes the contact between a fluid and an admixture, may be used. Additional examples include the generation of an aerosol through atomization or nebulization.

The interface-time within a gas-fluid contacting device is measurable, controllable, calculable and reportable. Furthermore, the interface-time may be for duration of up to 720 minutes, generally however, for duration of up to 120 minutes. Following the interface-time, the fluid exits the gas-fluid contacting device containing the quantifiable absorbed-dose of ozone. The elapsed-time, a composite of both the interface-time and the time for circulation of a fluid through other elements of an ozone delivery system is also measurable, controllable, calculable and reportable. This elapsed-time is for duration of up to 120 hours.

The pressure at the interface between fluid and ozone/oxygen admixture within a gas-fluid contacting device may be measured. Measurement of pressure within the device may be accomplished through the use of a pressure sensor inserted at the pressure port of the gas-fluid contacting device. The pressure at which an ozone/oxygen admixture interfaces with a fluid ranges from ambient pressure to 50 psi and may be performed between ambient pressure and 3 psi.

The temperature within a gas-fluid contacting device may be controlled by housing the device such that the connecting tubing containing both gas and fluid and an optional reservoir are maintained in a controlled temperature environment. A flow hood that provides for temperature regulation is an example of a controlled temperature environment. Alternatively, the addition of heating or cooling elements to the gas-fluid contact device may provide for the control of temperature. Measurement of temperature within the device may be accomplished through the use of a temperature sensor inserted at the temperature port of a gas-fluid contacting device. The temperature at which ozone/oxygen admixtures interface fluids ranges from 4° C. to 100° C., and may be performed at ambient temperature, 25° C., for example.

Gas-fluid contacting devices may be utilized individually or in conjunction with other such devices, whether they are similar or dissimilar in construction, design or orientation. In the event that multiple devices are utilized, either of the same design, or a combination of different gas-fluid contacting devices of different designs, these devices may be arranged one after the other in succession (in series), making a single device out of multiple individual contact devices.

In a series configuration of devices, a fluid flowing through the different contact devices flows in series, from the fluid exit port of one contact device to the fluid entrance port of the next, until passing through all the devices. The ozone/oxygen admixture may flow in a number of arrangements. In one example, the ozone/oxygen admixture flows through different contact devices in series, from the admixture exit port of one contact device to the admixture entrance port of the next. As an alternative example, the ozone/oxygen admixture may flow directly from the admixture source to the entrance port of each different contact device. Another alternative is a combination of the foregoing examples where the ozone/oxygen admixture flows from the exit port of some devices to the entrance port of other devices and in addition, to the entrance of some devices directly from the admixture source. In the event that multiple devices are utilized, the resultant fluid from the terminal device can either be collected or returned to the original device and recirculated.

When arranged in series with other contact devices, interface time between the fluid and ozone/oxygen admixture is controllable, and can be adjusted based on the individual pitch chosen for each device in series, or by adding additional devices to the series. The overall interface surface area will range from 0.01 m² for an individual device, and upwards based on the number of devices serially utilized.

### Example 1

An example of data measured and calculated by the ozone delivery system that utilizes a fluid target described herein is included in Table 1. Newborn Calf Serum commercially obtained was utilized as the target fluid. A variable pitch device with variable pitch platform, as disclosed in U.S. Pat. No. 7,736,494, was employed as the gas-fluid contacting device. The following initial conditions were utilized; 300 ppmv ozone inlet concentration, 145 ml initial fluid volume, 1000 ml per minute gaseous flow rate, 189 ml per minute fluid flow rate counter current to the ozone/oxygen admixture flow. Incremental reductions in fluid volume are due to sampling of fluid through the fluid access port.

TABLE 1
NEWBORN CALF SERUM
MEASURED VARIABLES

| | | | | Average Inlet Ozone | Average Exit Ozone |
|---|---|---|---|---|---|
| Elapsed-time | Fluid Volume | Gas Flow Rate | Fluid Flow Rate | Concentration | Concentration |
| (5 min intervals) | (milliliters) | (liters/minute) | (liters/minute) | (ppmv) | (ppmv) |
| 5 | 145 | 0.998 | 0.189 | 305.2 | 38.2 |
| 10 | 143 | 0.979 | 0.189 | 361.5 | 40.4 |
| 15 | 141 | 1.000 | 0.189 | 312.7 | 20.6 |
| 20 | 139 | 1.000 | 0.189 | 314.0 | 37.3 |

CALCULATED VARIABLES

| | Average | | | | |
|---|---|---|---|---|---|
| | Differential Ozone | | | Ozone-Absorbed per | Absorbed-dose |
| Elapsed-time | Concentration | Delivered-ozone | Residual-ozone | Interval | of Ozone |
| (minutes) | (ppmv) | (ug) | (ug) | (ug) | (ug) |
| 5 | 267.0 | 3.26E+03 | 4.08E+02 | 2.86E+03 | 2.86E+03 |
| 10 | 321.1 | 7.02E+03 | 8.28E+02 | 3.34E+03 | 6.20E+03 |
| 15 | 292.1 | 1.04E+04 | 1.06E+03 | 3.12E+03 | 9.32E+03 |
| 20 | 276.7 | 1.37E+04 | 1.46E+03 | 2.96E+03 | 1.23E+04 |

### Example 2

An additional example of data measured and calculated by the system described herein is in Table 2 below. Newborn Calf Serum commercially obtained was utilized as the target fluid. The variable pitch device with variable pitch platform, as disclosed in U.S. Pat. No. 7,736,494, was employed as the gas-fluid contacting device. The following initial conditions were utilized; 600 ppmv ozone inlet concentration, 137 ml initial fluid volume, 1000 ml per minute gaseous flow rate, 189 ml per minute fluid flow rate counter current to the ozone/oxygen admixture flow. Incremental reductions in fluid volume are due to sampling of fluid through the fluid access port.

TABLE 2
NEWBORN CALF SERUM
MEASURED VARIABLES

| | | | | Average Inlet Ozone | Average Exit Ozone |
|---|---|---|---|---|---|
| Elapsed-time | Fluid Volume | Gas Flow Rate | Fluid Flow Rate | Concentration | Concentration |
| (5 minute intervals) | (milliliters) | (liters/minute) | (liters/minute) | (ppmv) | (ppmv) |
| 5 | 137 | 1.000 | 0.189 | 604.2 | 72.0 |
| 5 | 135 | 1.000 | 0.189 | 609.6 | 63.5 |
| 5 | 133 | 1.000 | 0.189 | 606.6 | 70.8 |
| 5 | 131 | 1.000 | 0.189 | 605.3 | 71.7 |

CALCULATED VARIABLES

| | Average | | | | |
|---|---|---|---|---|---|
| | Differential Ozone | | | Ozone Absorbed | Absorbed-dose |
| Elapsed-time | Concentration | Delivered-ozone | Residual-ozone | per Interval | of ozone |
| (minutes) | (ppmv) | (ug) | (ug) | (ug) | (ug) |
| 5 | 532.2 | 6.47E+03 | 7.70E+02 | 5.69E+03 | 5.69E+03 |
| 10 | 546.1 | 1.30E+04 | 1.45E+03 | 5.84E+03 | 1.15E+04 |
| 15 | 535.8 | 1.95E+04 | 2.21E+03 | 5.73E+03 | 1.73E+04 |
| 20 | 533.6 | 2.60E+04 | 2.98E+03 | 5.71E+03 | 2.30E+04 |

### Example 3

Another example of data measured and calculated by the system described herein is in Table 3 below. Newborn Calf Serum commercially obtained was utilized as the target fluid. The variable pitch device, as disclosed in U.S. Pat. No. 7,736,494, was employed as the gas-fluid contacting device. The following initial conditions were utilized; 900 ppmv ozone inlet concentration, 145 ml initial fluid volume, 1000 ml per minute gaseous flow rate, 189 ml per minute fluid flow rate counter current to the ozone/oxygen admixture flow. Incremental reductions in fluid volume are due to sampling of fluid through the fluid access port.

TABLE 3
NEWBORN CALF SERUM
MEASURED VARIABLES

| | | | | Average Inlet Ozone | Average Exit Ozone |
|---|---|---|---|---|---|
| Elapsed-time | Fluid Volume | Gas Flow Rate | Fluid Flow Rate | Concentration | Concentration |
| (5 minute intervals) | (milliliters) | (liters/minute) | (liters/minute) | (ppmv) | (ppmv) |
| 5 | 145 | 1.000 | 0.189 | 908.1 | 68.0 |
| 5 | 143 | 1.000 | 0.189 | 911.4 | 50.1 |
| 5 | 141 | 1.000 | 0.189 | 904.4 | 46.6 |
| 5 | 139 | 1.000 | 0.189 | 904.7 | 50.9 |

CALCULATED VARIABLES

| | Average | | | | |
|---|---|---|---|---|---|
| | Differential Ozone | | | Ozone Absorbed | Absorbed-dose |
| Elapsed-time | Concentration | Delivered-ozone | Residual-ozone | per Interval | of ozone |
| (minutes) | (ppmv) | (ug) | (ug) | (ug) | (ug) |
| 5 | 840.1 | 9.72E+03 | 7.28E+02 | 8.99E+03 | 8.99E+03 |
| 10 | 861.3 | 1.95E+04 | 1.26E+03 | 9.22E+03 | 1.82E+04 |
| 15 | 857.8 | 2.92E+04 | 1.76E+03 | 9.18E+03 | 2.74E+04 |
| 20 | 853.8 | 3.88E+04 | 2.31E+03 | 9.13E+03 | 3.65E+04 |

In one embodiment of the invention, a method is provided to treat cardiovascular diseases in a mammal. The method involves subjecting an amount of blood, blood fractionate or other biological fluid, ex vivo, to an amount of ozone delivered by an ozone delivery system, as previously described. The method may also provide for the maintenance of the biological integrity of the treated fluid. The method provides treatment conditions for cardiovascular diseases at temperatures compatible with maintaining the biological integrity of biological fluids.

For blood products, the biological integrity of plasma may be measured by the functionality of its protein components either in whole plasma or after separation into plasma fractions. The biological integrity of red blood cell and platelet preparations may be determined by the methods and criteria known by those skilled in the art and are similar to those used in establishing the suitability of storage and handling protocols. In practical terms, the biological integrity of a biological fluid is a fluid that, subsequent to the method of treating cardiovascular diseases described herein, has sufficiently maintained its functionality upon re-infusion into a mammalian patient.

Fluid-contacting surfaces, including gas-fluid contacting devices constructed from ozone-inert material, may be treated with a human serum albumin (HSA) solution to prevent platelet adhesion, aggregation and other related platelet phenomena in the instances when a biological fluid to be treated contains platelets (i.e. whole blood, platelet concentrates). Generally, HSA solutions ranging between 1 and 10% may be employed. An HSA solution prepared in a biocompatible bacteriostatic buffer solution will be passaged throughout the gas-fluid contacting device. Subsequent to passage, the HSA solution will be drained from the device. The gas-fluid contacting device and all surfaces that are in contact with the biological fluid during the method described are consequently primed for use with platelet-containing biological fluids.

The present methods provide treatment of blood, blood fractionate or other fluid with a quantifiable absorbed dose of ozone to produce treated fluid that are then useful in the therapeutic treatment of cardiovascular diseases in mammals by administration of the treated blood, blood fractionate or other fluid to the patient.

In one or more embodiments of the invention, an aliquot of a mammalian patient's blood, or the separated cellular fractions of the blood, or mixtures of the separated cells, including platelets, is extracorporeally subjected to a measured amount of ozone such that it absorbs a quantifiable absorbed-dose of ozone. On reintroduction of this autologous aliquot to the patient's body, the blood, blood fractionate or other fluid that has been treated with a quantifiable absorbed-dose of ozone is used to promote and produce beneficial effects in the treatment of cardiovascular diseases. Reintroduction of this treated autologous aliquot may be through a variety of routes including intravenous, intramuscular and subcutaneous, or any combination thereof.

In another embodiment of the invention, the methods are directed to causing sufficient leukocyte apoptosis necessary to elicit clinical benefit when reinfused autologously into a patient. The methods may further cause sufficient leukocyte apoptosis without excessive necrosis necessary to elicit clinical benefit when reinfused autologously into a patient.

In certain embodiments of the invention, the autologous reinfusion of a patient's own blood or other body fluids provide therapeutic treatment by causing a reduction in CRP sufficient to elicit clinical benefit.

The methods of the present invention may comprise connecting a subject to a device for withdrawing blood, withdrawing blood containing blood cells from the subject, separating a non-cellular fraction from the blood and delivering a measured amount of ozone to the fraction under conditions which maintain the biological integrity of the blood fraction. The treated fraction is subsequently recombined with the blood cells and re-infused into the subject.

In another embodiment of the invention, therapeutic treatments for cardiovascular diseases and vascular disorders associated with deficient endothelial function, such as vasospastic disorders, may elicit a reduction of inflammation when reinfused autologously into a patient by reducing proinflammatory cytokines (e.g. interferon-gamma, TNF-gamma, IL-6, IL-8 and IL-12) and/or an increase in anti-inflammatory cytokines (e.g. interleukin-4 and IL-10) released by immunomodulatory T cells.

The methods of the present invention are directed to therapeutic treatment of cardiovascular disease and related conditions by reinfusing biological fluids treated in accordance with the methods described herein to elicit a reduction of inflammation when reinfused autologously into a patient, resulting in any number of clinical benefits, including improvement in blood flow which yields enhanced oxygenation.

The therapeutic effects derived from the treatment and reinfusion of blood, blood fractionate or other fluid which have absorbed a quantifiable absorbed-dose of ozone in accordance with the methods described herein include changes in lipid metabolism and enhancement of the immune system through stimulation of leukocytes (i.e. cell-cell interaction or cytokine release) throughout the peripheral blood of the patient leading to reduction in atherosclerotic plaque formation and deposition. These effects may result in a reversal in progressive luminal narrowing in arteries, a reduction in the rupture or denudation of plaques decreasing the incidence of atheroembolism (cholesterol embolism), and improved blood flow yielding enhanced oxygenation.

Regarding disorders involving atherosclerotic plaque formation, deposition and rupture, the present methods provide therapeutic treatment and prophylaxis of a wide variety of such mammalian disorders including cardiovascular diseases, such as atherosclerosis, peripheral arterial occlusive disease, cerebrovascular disease (stroke and transient ischemic attack), myocardial infarction, angina, hypertension, retinal ischemia, renal failure, abdominal aortic aneurysm, and hyperlipidemia.

Further, treatment and reinfusion of biological fluids in accordance with the methods described herein provide therapeutic treatment of cardiovascular disease, including vascular disorders associated with deficient endothelial function, such as vasospastic disorders. The present methods provide for therapeutic stimulation of the activity of a functionally deficient endothelium.

Other beneficial effects that may derive from the methods of the present invention, as described herein, include reduction of edema, which may be brought about by inducing leukocyte apoptosis without excessive necrosis. Additional benefits include reduction of CRP to elicit an anti-inflammatory response, and the promotion of blood flow to ischemic tissue. The effectiveness of blood flow to ischemic tissue brought about by the therapeutic methods of the present invention may be evaluated by a variety of diagnostic tools including MRI, CT perfusion and Doppler imaging techniques.

Therapeutic treatment and reinfusion of biological fluids in accordance with the methods described herein are further directed to enhancement of the immune system through stimulation of leukocytes (i.e. cell-cell interaction or cytokine release) throughout the peripheral blood of the patient leading to improved blood flow, increase in vasodilation (i.e. promotion of vasodilators or inhibition of vasoconstrictors), improvement in endothelial function including endothelial cellular repair or replacement and enhanced oxygenation. The present methods provide for the therapeutic treatment and prophylaxis of a wide variety of such mammalian disorders, including cardiovascular diseases such as atherosclerosis, peripheral arterial occlusive disease, congestive heart failure, cerebrovascular disease (stroke), myocardial infarction, angina, hypertension, vasospastic disorders such as Raynaud's disease, cardiac syndrome X, and migraine.

The methods of the present invention may include treatment of blood or other biological fluids from a mammalian patient by a discontinuous flow method where blood is withdrawn from the patient using a device suitable for withdrawing blood, separating a non-cellular fraction from the blood and delivering a measured amount of ozone to the fraction under conditions which maintain the biological integrity of the blood fraction, recombining the blood cells with the blood and reinfusing the treated blood into the patient. In those aspects of the invention where the method of treatment involves a discontinuous approach, the volume of blood removed can range from 1 to 5000 ml, depending on patient size and blood volume. This discontinuous treatment approach may be performed once or multiple consecutive times during a single treatment.

Methods of the present invention also include removing blood directly from a subject and re-infusing it to the same subject or patient in a continuous loop configuration. The blood may circulate through the loop, which includes the gas-fluid contacting device, once or multiple times, wherein a measured amount of ozone is delivered to the blood under conditions which maintain the biological integrity of the blood. The treated blood is constantly being re-infused directly back into the same patient.

Methods of the present invention are further effective in providing therapeutic treatment of blood and biological fluids to reduce cholesterol, triglycerides and other lipids in a mammalian patient. Such methods comprise removing blood directly from a subject and reinfusing it to the same patient in a continuous loop configuration.

In those aspects of the invention where the method of treatment involves a continuous loop approach, the volume of blood treated can range between can vary between 10 ml and the total estimated circulating blood volume of a mammalian patient being treated multiple times. Generally, the blood volume treated will range between 10 ml and 10000 ml and preferably range between 10 ml and 6000 ml.

The time required for an individual treatment through the use of a continuous loop format is based on a number of factors including the desired number of passes through the loop, volume of the fluid treated, the flow rate at which the fluid is circulating, the interface time required between the fluid and the amount of delivered-ozone, and the amount of the quantifiable absorbed-dose of ozone required. The time for the treatment can range from 1 minute to 720 minutes and preferably range from 1 minute to 180 minutes.

The number and frequency of treatments can vary considerably based upon the clinical situation of a particular patient. Generally the number of treatments can range between an individual treatment and 200 treatments, to be provided on a daily, alternate day or other schedule based on the clinical evaluation of the patient and desired clinical outcomes. Upon completion of a number of treatments and evaluation by a health care practitioner, another course of treatments may be indicated.

Alternative applications of the present methods involve plasmapheresis, wherein the patient's plasma is selectively removed while the balance of the blood cells is immediately returned to the patient. A measured amount of ozone is delivered to the isolated plasma under conditions which may maintain the biological integrity of the plasma. The treated plasma is subsequently re-infused into the subject.

The methods of the present invention are described for treatment of conditions attendant to cardiovascular disease and related condition, comprising methods that employ ozone delivery devices that are constructed with all ozone-contacting surfaces being made or constructed of ozone-inert materials to assure accurate determination of the amount of ozone delivered to a fluid being treated, and to assure accurate determination of the amount of ozone absorbed by the fluid. The ozone delivery structures and related methods to treat blood and other biological fluids with ozone, and the use of those fluids for therapeutic treatments as disclosed herein, may be varied from those described to adapt them to specific applications. Therefore, reference to specific constructions and methods of use are by way of example and not by way of limitation.

What is claimed is:

1. A method of treating cardiovascular disease and related conditions in a subject suffering from, or believed to be suffering from, cardiovascular disease, comprising:
providing a biological fluid withdrawn from a mammalian subject;
processing said fluid in an ozone delivery system to deliver to said fluid a measured amount of ozone to effect absorption by the fluid of a quantifiable absorbed dose of ozone; and
reintroducing the treated fluid having a quantifiable absorbed dose of ozone to the mammalian subject to provide therapeutic treatment of cardiovascular disease and related conditions and symptoms.

2. The method according to claim 1, wherein said processing of the fluid is carried out in an ozone delivery system all gas-contacting surfaces of which are constructed of ozone-inert materials.

3. The method according to claim 1, wherein said processing of the fluid is carried out in a discontinuous loop format.

4. The method according to claim 1, wherein said processing of the fluid is carried out in a continuous flow format.

5. The method according to claim 1, wherein said biological fluid is blood, a blood derivative or a blood fractionate.

6. The method according to claim 5, wherein said blood fractionate comprises separated cellular fractions or platelets.

7. The method according to claim 1, wherein said processing of said biological fluid is carried out in a manner to maintain the biological integrity of said fluid.

8. The method according to claim 1, wherein said biological fluid is blood and said treatment is conducted as plasmapheresis, further comprising;
isolating a portion of plasma from the blood;
subjecting the isolated plasma to a measured amount of ozone to effect absorption by the plasma of a quantifiable absorbed dose of ozone; and
reinfusing the plasma containing a quantifiable absorbed dose of ozone to the subject.

9. The method according to claim 1, wherein the effects of the therapeutic treatment comprise reduction in atherosclerotic plaques, regression in atherosclerotic plaque formation, reduction in triglycerides, cholesterol and other lipids or reduction in lipid deposits, and combinations thereof.

10. The method according to claim 1, wherein the treatment of the subject's blood produces therapeutic improvement in one or more cardiovascular disease conditions and related conditions, including atherosclerosis, peripheral arterial occlusive disease, cerebrovascular accident, angina pectoris, vasospastic disorders, Raynaud's disease, dyslipidemia, congestive heart failure and hypertension.

11. The method according to claim 1, wherein the effects of the therapeutic treatment comprise reduction in edema, reduction of inflammation, improvement in blood flow or reduction in episodes of claudication, and combinations thereof.

12. A method of producing a therapeutic substance for the treatment of cardiovascular disease and related symptoms or conditions thereof, comprising:
providing a biological fluid;
delivering to the biological fluid a measured amount of ozone to produce a therapeutic substance having a quantifiable absorbed-dose of ozone which, upon administration to a subject suffering, or believed to be suffering, from cardiovascular disease, effective treats the symptoms and conditions related to the cardiovascular disease.

13. The method according to claim 12, wherein said biological fluid is blood, a blood derivative or blood fractionate.

14. A medicament for the treatment of acute ischemic brain stroke, and the symptoms or conditions related thereto, comprising a biological fluid containing a quantifiable absorbed-dose of ozone to provide efficacious therapeutic effect to a subject suffering, or believed to be suffering, from acute ischemic brain stroke and the symptoms or conditions related thereto upon administration of the medicament to the subject.

15. The medicament according to claim 14, wherein said biological fluid is blood, a blood derivative or blood fractionate.

15. The medicament according to claim 15, wherein said blood fractionate is comprised of platelets.

16. The medicament according to claim 15, wherein said blood derivative is plasma.
